(19) United States
(12) Patent Application Publication
(10) Pub. No.: US 2006/0229613 A1
(43) Pub. Date: Oct. 12, 2006

(54) Sheath assembly for spinal stabilization device

(76) Inventors: Jens P. Timm, West Haven, CT (US); John Anthony, Corning, NY (US)

Correspondence Address: Basam E. Nabulsi;McCARTER & ENGLISH, LLP, Four Stamford Plaza, 107 Elm Street, Stamford, CT, 06902, US

(21) Appl. No.: 11/332,460

(22) Filed: Jan. 13, 2006

Related U.S. Application Data
(63) Continuation-in-part of application No. 11/027,073, filed on Dec. 31, 2004.

(51) Int. Cl. A61F 2/30

(52) U.S. Cl. 606/61

(57) ABSTRACT

Systems, assemblies and methods for assembling a sheath member with respect to a spinal stabilization device are provided. The disclosed sheath assembly includes at least one connector ring mounted with respect to a sheath member. The connector ring defines an inner wall that shields the sheath member from elements positioned therewithin, e.g., spring members, and generally includes a plurality of radially-spaced notches. The connector ring receives the sheath member within an internal cavity and is secured thereto by crimping, compression or swaging. The sheath assembly may be mounted with respect to a spinal stabilization device by crimping, compressing or swaging the connector ring to an underlying structure, e.g., an end cap or associated flange. The internal cavity of the connector ring is generally defined by an inner face, an outer wall and an intermediate apex region. Methods for assembly are also disclosed.

### CROSS-REFERENCE TO RELATED APPLICATIONS

The present application is a continuation-in-part of a co-pending, commonly assigned U.S. patent application entitled “Surgical Implant Devices and Systems Including a Sheath Member,” which was filed on Dec. 31, 2004 and assigned Ser. No. 11/027,073. The entire contents of the foregoing patent application are incorporated herein by reference.

### BACKGROUND

1. Technical Field

The present disclosure is directed to a system and method for assembling a sheath with respect to a spinal stabilization device. More particularly, the present disclosure is directed to a sheath subassembly that includes at least one connector ring mounted with respect to a sheath member, and methods for fabricating the sheath subassembly and subsequently mounting the sheath subassembly with respect to a spinal stabilization device.

2. Background Art

Low back pain is one of the most expensive diseases afflicting industrialized societies. With the exception of the common cold, it accounts for more doctor visits than any other ailment. The spectrum of low back pain is wide, ranging from periods of intense disabling pain which resolve to varying degrees of chronic pain. The conservative treatments available for lower back pain include: cold packs, physical therapy, narcotics, steroids and chiropractic maneuvers. Once a patient has exhausted all conservative therapy, the surgical options generally range from micro discectomy, a relatively minor procedure to relieve pressure on the nerve root and spinal cord, to fusion, which takes away spinal motion at the level of pain.

Each year, over 200,000 patients undergo lumbar fusion surgery in the United States. While fusion is effective about seventy percent of the time, there are consequences even to these successful procedures, including a reduced range of motion and an increased load transfer to adjacent levels of the spine, which may accelerate degeneration at those levels. Further, a significant number of back-pain patients, estimated to exceed seven million in the U.S., simply endure chronic low-back pain, rather than risk procedures that may not be appropriate or effective in alleviating their symptoms.

Spinal stabilization devices have been developed to provide relief to individuals suffering from lower back pain. Spinal stabilization devices frequently extend from a first pedicle screw to a second pedicle screw, and may include one or more rigid rods to provide a stabilizing force to the treated spinal region. New treatment modalities, collectively called motion preservation devices, are currently being developed, such as nucleus, disc or facet replacements. Other motion preservation devices provide dynamic internal stabilization of the injured and/or degenerated spine, without removing any spinal tissues, e.g., the Dynesys stabilization system (Zimmer, Inc.; Warsaw, Ind.) and the Graf Ligament. A major goal of these devices/systems is stabilization of the spine to prevent pain while preserving near normal spinal function. The primary difference in the two types of motion preservation devices is that replacement devices are utilized with the goal of replacing degenerated anatomical structures which facilitate motion while dynamic internal stabilization devices are utilized with the goal of stabilizing and controlling abnormal spinal motion.

Over ten years ago a hypothesis of lower back pain was presented in which the spinal system was conceptualized as consisting of the spinal column (vertebrae, discs and ligaments), the muscles surrounding the spinal column, and a neuromuscular control unit which helps stabilize the spine during various activities of daily living. Panjabi M M. “The stabilizing system of the spine. Part I. Function, dysfunction, adaptation, and enhancement.” J Spinal Disord 5 (4): 383-389, 1992a. A corollary of this hypothesis was that strong spinal muscles are needed when a spine is injured or degenerated. This was especially true while standing in neutral posture. Panjabi M M. “The stabilizing system of the spine. Part II. Neutral zone and instability hypothesis.” J Spinal Disord 5 (4): 390-397, 1992b. In other words, a low-back patient needs to have sufficient well-coordinated muscle forces, strengthening and training the muscles where necessary, so they provide maximum protection while standing in neutral posture.

Dynamic stabilization (non-fusion) devices need certain functionality in order to assist the compromised (injured or degenerated with diminished mechanical integrity) spine of a back patient. Specifically, the devices must provide mechanical assistance to the compromised spine, especially in the neutral zone where it is needed most. The “neutral zone” refers to a region of low spinal stiffness or the toe-region of the Moment-Rotation curve of the spinal segment (see FIG. 1). Panjabi M M, Goel V K, Takata K. 1981 Volvo Award in Biomechanics. “Physiological Strains in Lumbar Spinal Ligaments, an in vitro Biomechanical Study.” Spine 7 (3): 192-203, 1982. The neutral zone is commonly defined as the central part of the range of motion around the neutral posture where the soft tissues of the spine and the facet joints provide least resistance to spinal motion.

Experiments have shown that after an injury to the spinal column and/or degeneration of the spine, neutral zones, as well as ranges of motion, increase. However, the neutral zone increases to a greater extent than does the range of motion, when described as a percentage of the corresponding intact values. This implies that the neutral zone is a better measure of spinal injury and instability than the range of motion. Clinical studies have also found that the increase in range of motion does not correlate well with low back pain. Therefore, an unstable spine needs to be stabilized, especially in the neutral zone.

Dr. Panjabi discloses an advantageous dynamic spine stabilizer in U.S. Patent Publication No. 2004/0236329, the entire contents of which are incorporated herein by reference. The disclosed dynamic spine stabilizer generally includes a support assembly in the form of a first housing member and a second housing member that are telescopically connected. According to exemplary embodiments of the Panjabi disclosure, first and second springs are positioned between the housing members, and spring compression may be set by adjusting the relative distance between the first and second housing members. Although springs are employed in accordance with a preferred embodiment of the Panjabi disclosure, the use of other elastic members is also contemplated. A piston assembly links the first spring and the second spring to first and second ball joints associated with pedicle screws. The piston assembly generally includes a piston rod and retaining rods that cooperate with the first and second springs. The disclosed Panjabi devices/systems offer significantly enhanced spinal stabilization. See also U.S. Patent Publication No. 2005/0245930 to Timm and Panjabi, the entire contents of which are incorporated herein by reference.

In the noted Timm and Panjabi application to which the present application claims priority (U.S. Patent Publication No. 2005/0245930), a surgical implant is provided that includes first and second abutment surfaces between which are positioned a force imparting mechanism. A sheath is positioned between the first and second abutment surfaces, and surrounds the force imparting mechanism. The sheath is fabricated from a material that accommodates relative movement of the abutment members, while exhibiting substantially inert behavior relative to surrounding anatomical structures. The sheath is generally fabricated from expanded polytetrafluoroethylene, ultra-high molecular weight polyethylene, a copolymer of polycarbonate and a urethane, or a blend of a polycarbonate and a urethane. The force imparting member may include one or more springs, e.g., a pair of nested springs. The surgical implant may be a dynamic spine stabilizing member that is advantageously incorporated into a spine stabilization system to offer clinically efficacious results.

Despite efforts to date, a need remains for efficacious spinal stabilization devices that provide desired levels of stabilization and that exhibit clinically acceptable interaction with surrounding anatomical elements and structures. More particularly, a need remains for spinal stabilization devices that provide dynamic spinal stabilization while protecting against undesirable interaction between the dynamic force-imparting element(s) and the surrounding anatomy. Still further, a need remains for fabrication and/or assembly methods for manufacture of spinal stabilization devices, including particularly dynamic spinal stabilization devices, in a reliable and efficacious manner. These and other needs are satisfied by the disclosed spinal stabilization devices/systems and associated assembly methods.

### SUMMARY OF THE DISCLOSURE

Spinal stabilization systems and/or other surgical implants that include a cover and/or sheath structure are desirable in that they provide protection to inner force-imparting component(s), e.g., one or more springs, while exhibiting clinically acceptable interaction with surrounding anatomical fluids and/or structures. According to the present disclosure, a sheath member is provided for positioning with respect to a stabilization device, e.g., a spinal stabilization device. The sheath member is mounted with respect to at least one, and generally a pair of connector rings.

According to exemplary embodiments of the present disclosure, a sheath assembly is provided that includes a sheath member and at least one connector ring secured with respect to the sheath member. The connector ring generally defines a circumference and includes a plurality of radially-spaced notches positioned around such circumference. The sheath member is typically substantially cylindrical in geometry, and may be advantageously fabricated from expanded polytetrafluoroethylene (ePTFE), ultra-high molecular weight polyethylene, a copolymer of polycarbonate and a urethane, and a blend of a polycarbonate and a urethane.

Exemplary connector rings according to the present disclosure define a substantially V-shaped cross-section before being secured to the sheath member. The radially-spaced notches include an aperture region and a notch finger. Exemplary connecting rings also include an inner face, an apex region and an outer wall. The radially-spaced notches are generally formed in the apex region (in whole or in part). The inner face may define a deflected edge which, in preferred embodiments, is substantially parallel to the outer wall. The inner face advantageously spaces or shields the inner wall of the sheath member from dynamic member(s) that are positioned therewithin, thereby avoiding potentially undesirable wear or abrasion.

A pair of connector rings may be provided, with a first connector ring being secured to a first end of the sheath member and a second connector ring being secured to the second end of the sheath member. Generally, the connector ring defines an interior cavity and an end of the sheath member is positioned in the internal cavity prior to being secured thereto, e.g., by crimping, compression or swaging.

In further embodiments of the present disclosure, a spinal stabilization device is provided that includes first and second end caps in a spaced relation, at least one spring member positioned between the first and second end caps; and a sheath assembly mounted with respect to the first and second end caps and around the at least one spring member. The sheath assembly generally includes a sheath member and at least one connector ring secured with respect to the sheath member, the at least one connector ring defining a circumference and including a plurality of radially-spaced notches positioned around the circumference. As noted previously, the sheath member is typically fabricated from an inert material, e.g., expanded polytetrafluoroethylene (ePTFE), ultra-high molecular weight polyethylene, a copolymer of polycarbonate and a urethane, and a blend of a polycarbonate and a urethane. The sheath assembly may be advantageously secured with respect to the first and second end caps by crimping, compression or swaging.

The present disclosure further provides a method for assembling a sheath assembly that includes: (i) providing a sheath member; (ii) providing a first connector ring that includes an inner face, an apex region, an outer wall and a plurality of radially-spaced notches formed at least in part in the apex region, wherein the first connector ring defines an internal cavity; (iii) positioning an end of the sheath member within the internal cavity; and (iv) securing the sheath member with respect to the first connector ring by crimping, compression or swaging.

The disclosed method may further include the additional steps of (i) providing a second connector ring that includes an inner face, an apex region, an outer wall and a plurality of radially-spaced notches formed at least in part in the apex region, wherein the second connector ring defines a second internal cavity; (ii) positioning an opposite end of the sheath member within the second internal cavity; and (iii) securing the sheath member with respect to the second connector ring by crimping, compressing or swaging. The sheath member may be secured with respect to the spinal stabilization device by crimping, compressing or swaging of the first connector ring with respect to the first end cap, and crimping, compressing or swaging of the second connector ring with respect to the second end cap.

These and other structural, functional and operational benefits of the present disclosure will be apparent from the detailed description which follows, particularly when read in conjunction with the appended figures.

### BRIEF DESCRIPTION OF THE FIGURES

To assist those of ordinary skill in the art in making and using the disclosed spinal stabilization devices, reference is made to the accompanying figures, wherein:

FIG. 1A is a side view of an exemplary spinal stabilization device according to the present disclosure;

FIG. 1B is a top view of the exemplary spinal stabilization device of FIG. 1A;

FIG. 2A is a top view of the exemplary spinal stabilization device of FIGS. 1A and 1B with the disclosed sheath assembly removed;

FIG. 2B is a cross-sectional view of the spinal stabilization device of FIG. 2A taken along line B-B;

FIG. 2C is an end view taken from the left end of the exemplary spinal stabilization device of FIGS. 1A-1B and 2A-2B;

FIG. 2D is an end view taken from the right end of the exemplary spinal stabilization device of FIGS. 1A-1B and 2A-2B;

FIG. 3A is a top plan view of an exemplary connector ring for use in assembling a sheath subassembly according to the present disclosure;

FIG. 3B is a bottom plan view of the exemplary connector ring of FIG. 3A;

FIG. 3C is a side view of the exemplary connector ring of FIGS. 3A and 3B:

FIG. 3D is an end view of the exemplary connector ring of FIGS. 3A-3C;

FIG. 3E is a sectional view of the exemplary connector ring of FIGS. 3A-3D taken along line E-E of FIG. 3D;

FIG. 4 is a detail sectional view of a portion of the connector ring of FIGS. 3A-3E;

FIG. 5A is a side view of an exemplary sheath subassembly according to the present disclosure;

FIG. 5B is a sectional side view of the exemplary sheath subassembly of FIG. 5A, taken along line B-B, according to the present disclosure;

FIG. 5C is a front view of the exemplary sheath subassembly of FIG. 5A; and

FIG. 6 is a side view of an exemplary stabilization device mounted with respect to a pair of pedicle screws according to the present disclosure.

### DESCRIPTION OF EXEMPLARY EMBODIMENT(S)

The present disclosure provides advantageous spinal stabilization systems and methods for assembling, fabricating and/or manufacturing such spinal stabilization systems. More particularly, the present disclosure provides advantageous sheath subassemblies that are configured and dimensioned to be mounted with respect to a spinal stabilization system. In exemplary embodiments of the present disclosure, the disclosed sheath subassemblies may be mounted with respect to dynamic element(s), e.g., spring member(s), that are adapted to provide dynamic stabilization to a spinal region, thereby encasing or otherwise enclosing the dynamic element(s) and protecting against potentially undesirable anatomical interaction between such dynamic element(s) and the surrounding anatomical elements/structures. Still further, the present disclosure provides advantageous methods and/or techniques for fabricating a sheath subassembly for assembly as part of a spinal stabilization device, e.g., a dynamic spinal stabilization device.

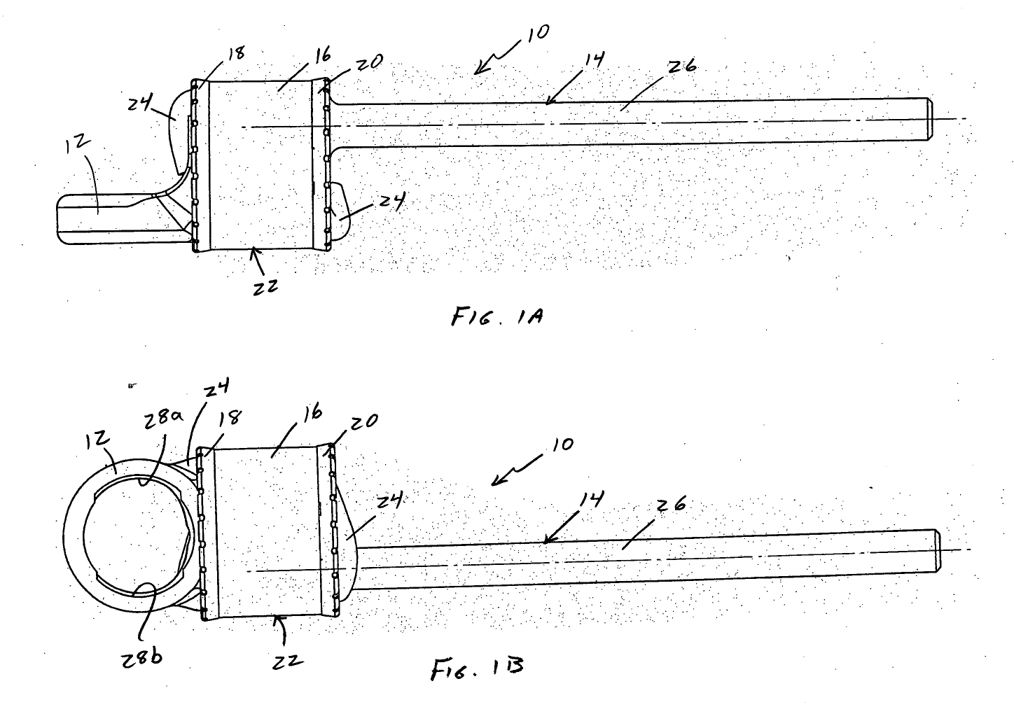

With initial reference to FIGS. 1A and 1B, an exemplary spinal stabilization device 10 is schematically depicted. Stabilization device 10 includes first end cap 12 and second end cap 14. Sheath member 16 is positioned between first and second end caps 12, 14, and surrounds, inter alia, spring elements (described below). First and second connector rings 18, 20 are secured with respect to sheath member 16 and, with sheath member, define a sheath assembly 22. Outer spring member 24 is exposed at both ends of sheath assembly 22. The design, operation and function of outer spring member 24 is described in greater detail below.

Second end cap 14 defines an elongated rod 26 that is generally of circular cross-section and that is adapted for mounting with respect to a pedicle screw (see, e.g., FIG. 6). First end cap 12 defines a substantially circular ring with opposed features 28a, 28b that facilitate interaction with cooperative structures/features on a pedicle screw mounting structure. For example, FIG. 6 (described in greater detail below) illustrates an exemplary mounting arrangement for first end cap 12 with respect to a pedicle screw. The overall size and configuration of spinal stabilization device 10 is such that stabilization of a single spinal level is achieved. Multiple spinal stabilization devices may be advantageously employed to stabilize multiple levels, e.g., adjacent spinal levels.

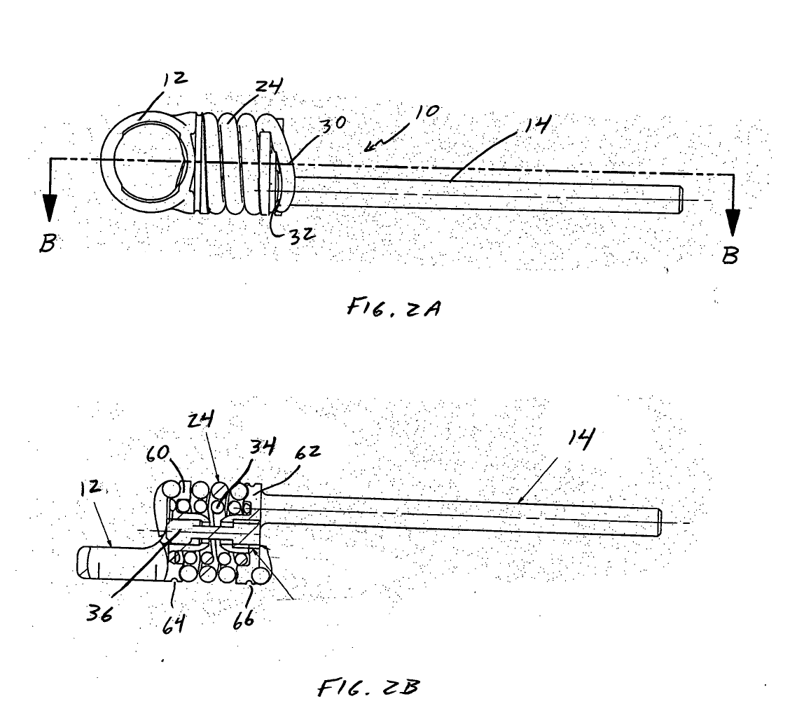

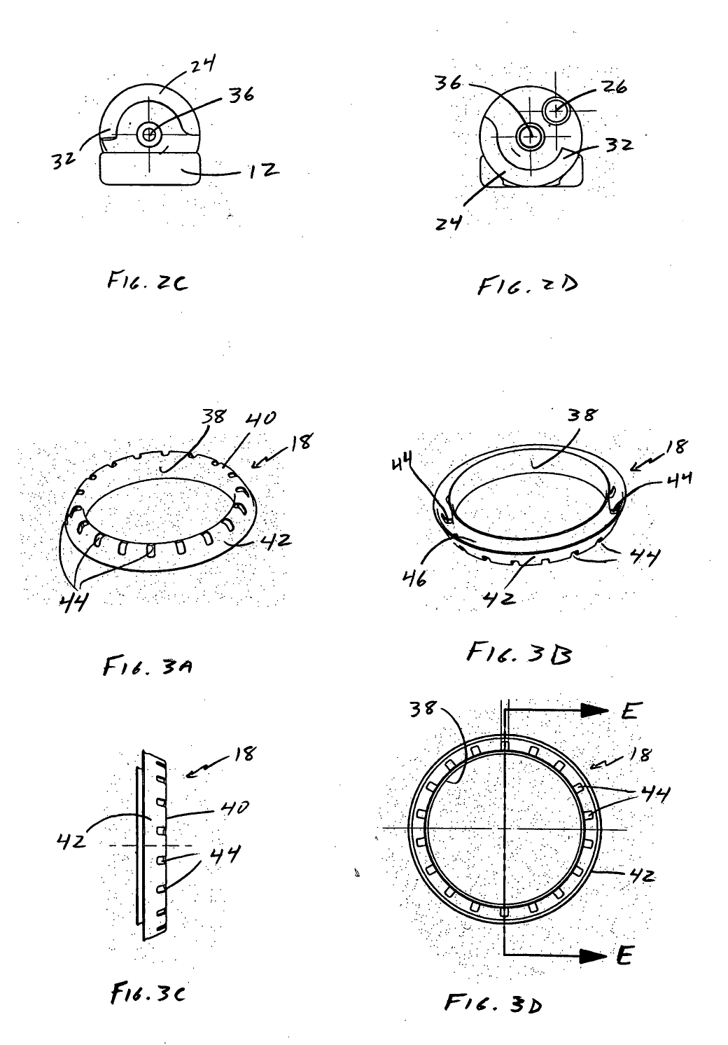

Turning to FIGS. 2A, a further view of spinal stabilization device 10 is provided, with sheath assembly 22 removed. As shown therein, spinal stabilization device 10 includes an outer spring member 24 that is mounted with respect to first and second end caps 12, 14. An advantageous method for mounting outer spring member 24 with respect to end caps 12, 14 is disclosed in a co-pending U.S. patent application entitled “Spring Junction and Assembly Methods for Spinal Device” (Ser. No. 11/196,102; filed Aug. 3, 2005), the contents of which are hereby incorporated by reference. Thus, in an exemplary assembly method according to the present disclosure, a spring junction is defined between outer spring member 24 and end caps 12, 14, and the spring junction includes a weld region 32. With reference to FIGS. 2A, 2C and 2D, a heat-affected zone is defined at either end of outer spring member 24, and is disposed adjacent weld region 32. However, the heat-affected zone is physically separately disposed with respect to the active region of spring member 24. Weld region 32 is subjected to a welding process, such as electron-beam welding, and accordingly may be exposed to welding temperatures of about 1000° F. or higher. Outer spring member 24 includes a bend region 30 disposed between weld region 32 and an adjacent coil of the spring member that extends along a helical path. Bend region 30 is generally sized and shaped so as to initially bend away from the spring member's helical path before bending back toward the helical path and terminating at or in weld region 32. Of note, the disclosed spring junctions are typically formed at both ends of outer spring member 24, thereby securing outer spring member 24 with respect to both end caps 12, 14.

With reference to FIG. 2B, a cross-sectional view of spinal stabilization device 10 taken along line B-B in FIG. 2A is provided. As shown in FIG. 2B, spinal stabilization device 10 includes a second, inner spring member 34 that is concentrically mounted within outer spring member 24. An internal cable assembly 36 is concentrically positioned within both spring members 24, 34, and is fixedly mounted with respect to end caps 12, 14. The design, assembly and operation of an exemplary cable assembly 36 is described in a co-pending U.S. patent application entitled “Dynamic Spine Stabilization Device with Travel-Limiting Functionality” (Ser. No. 11/189,512; filed Jul. 26, 2005), the contents of which are hereby incorporated by reference. As described in the foregoing co-pending patent application, internal cable assembly 36 advantageously functions to define and/or impose a maximum distance by which end caps 12, 14 may be separated, i.e., the relative travel therebetween.

Spring members 24, 34 cooperate to provide advantageously dynamic spinal stabilization. Indeed, the advantageous performance of exemplary embodiments of the disclosed spinal stabilization device 10 is described in a co-pending U.S. patent application entitled “Dynamic Spine Stabilizer” (Ser. No. 11/132,538; filed May 19, 2005), the contents of which are hereby incorporated by reference. Through the cooperative action of spring members 24, 34, the resistance of spinal stabilization device 10 is applied to a spinal region such that greater mechanical assistance is provided while the spine is around its “neutral zone” and lesser mechanical assistance is provided while the spine bends beyond its neutral zone. In exemplary embodiments, the disclosed spinal stabilization device 10 delivers a predetermined level of resistance, while accommodating a predetermined travel distance (i.e., linear travel) between adjacent pedicles, e.g., a predetermined level of resistance in the range of about 150 lbs/inch to about 450 lbs/inch and a predetermined travel distance of about 1.5 mm to about 5 mm.

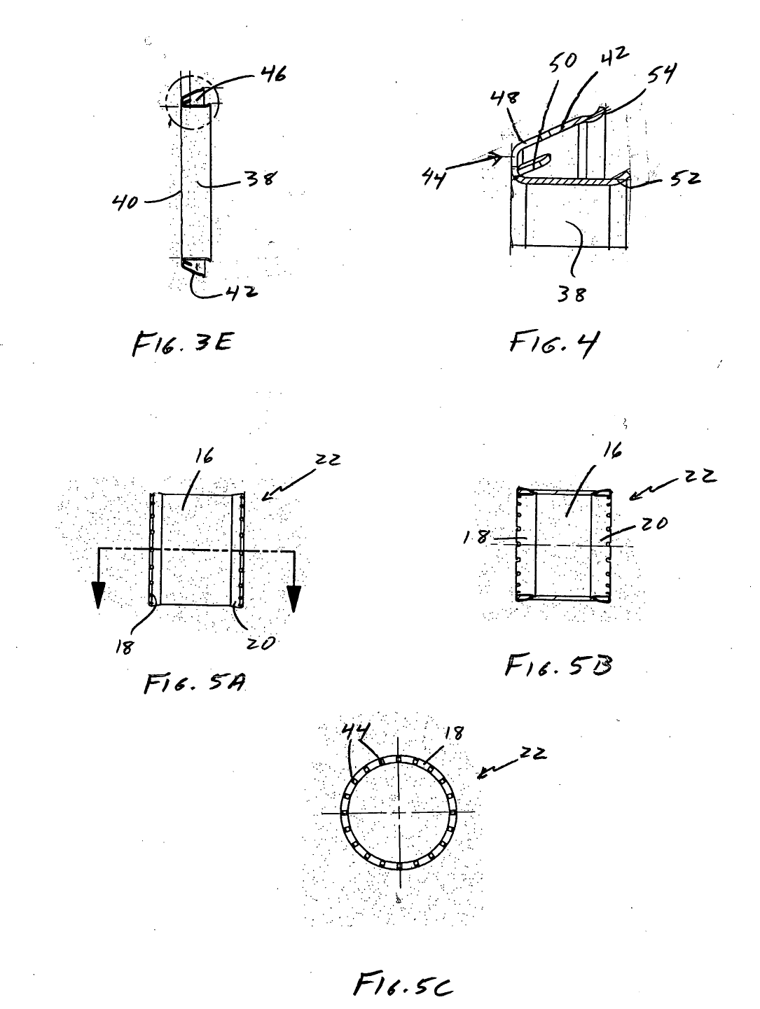

Turning to FIGS. 3A-3D, a series of views of an exemplary connector ring 18 according to the present disclosure are provided. Of note, connector rings 18, 20 are generally identical and interchangeable. Thus, the description provided herein with respect to connector ring 18 applies with equal force to connector ring 20. Connector ring 18 includes an inner cylindrical face 38, a substantially circular apex region 40, and an angularly oriented outer wall 42. A plurality of radially-spaced notches 44 are formed in the apex region 40 of connector ring 18. With reference to FIG. 3B, an internal cavity 46 is defined within connector ring 18 between cylindrical face 38 and outer wall 42.

FIGS. 3E and 4 provide more detailed views of exemplary connector ring 18. With initial reference to FIG. 3E, a cross-section view of connector ring 18 taken along line E-E in FIG. 3D is provided. As shown therein, connector ring 18 is characterized by a substantially V-shaped cross-section with apex region 40 defining the apex thereof. Spaced notches 44 generally do not extend into inner face 38, but are limited to the apex region 40 and, to a limited degree, downward extension to outer wall 42. In the disclosed exemplary embodiment, eighteen (18) radially-spaced notches 44 are provided. However, the present disclosure is not limited to such exemplary notch count; indeed, more or less notches may be defined on connector ring 18, without departing from the spirit or scope of the present disclosure. Indeed, it is contemplated that the number of radially-spaced notches may range, for example, from 12 to 30.

With reference to the detailed view of FIG. 4, which relates to the region shown within the dashed circle in FIG. 3E, additional structural details associated with an exemplary connector ring 18 according to the present disclosure are depicted. Notch 44 includes an aperture region 48 and a notch finger 50 which extends into internal cavity 46. Notch finger 50 is typically defined by notching or lancing the apex region 40 to define aperture region 48 and flex notch finger 50 inwardly. Radially-spaced notches 44 may be individually formed using an appropriate fixture/tool. Alternatively, a plurality of radially-spaced notches 44 may be formed simultaneously, e.g., by fixturing connector ring 18 and simultaneously notching/lancing notches 44 around the circumference thereof According to preferred embodiments of the present disclosure, all radially-spaced notches 44 are simultaneously formed around the circumference of connector ring 18.

With further reference to FIG. 4, inner face 38 defines a deflected edge 52 that extends toward internal cavity 46. Deflected edge 52 typically extends around the circumference of inner face 38 and, according to preferred embodiments of the present disclosure, defines a surface that is substantially parallel to outer wall 42. Deflected edge 52 is typically angled relative to inner face 38 at an angle of about 25°, although alternative angular orientations may be employed (including the omission of deflected edge 52) without departing from the spirit or scope of the present disclosure. Similarly, outer wall 42 is typically angled relative to inner face 38 at an angle of about 25°, although alternative angular orientations may be employed according to the present disclosure. Indeed, outer wall 42 need not be planar, but may be radiused, in whole or in part. As shown in FIG. 4, an inward depression 54 may be formed along the edge of outer wall 42, such inward depression 54 typically extending around the circumference of outer wall 42. Deflected edge 52 and inward depression 54 advantageously function to capture and/or retain a sheath member during the assembly process, as described in greater detail. Alternative and/or additional techniques may be employed to facilitate such assembly processes, e.g., an adhesive and/or tacky material may be provided in internal cavity 46.

Connector rings 18, 20 are typically fabricated from a metal material, e.g., stainless steel or titanium. The dimensions associated with connector rings 18, 20 will depend on the size and geometry of the spinal stabilization device with which they will be employed. However, in exemplary embodiments of the present disclosure, connector rings 18, 20 define an inner diameter (i.e., the diameter of inner face 38) of about 0.5 inches, although alternative geometries, e.g., diameters of about 0.4 to about 0.75 inches, are contemplated. The length of inner face 38 may range depending on the overall size and geometry of the spinal stabilization device, e.g., from about 0.075 to about 0.2 inches and, in exemplary embodiments of the present disclosure, the length of inner face is about 0.1 inch. The width and length of aperture regions 48 are typically selected so as not to risk the structural integrity of connector rings 18, 20, while simultaneously providing the advantageous mounting properties described below with reference to the assembly of sheath subassemblies. However, in exemplary embodiments of the present disclosure, the aperture regions 48 are approximately 0.025 inches in width and approximately 0.06 inches in length, although alternative dimensions, e.g., a width of between about 0.015 and 0.04 inches and a length of about 0.04 to about 0.125 inches, are contemplated.

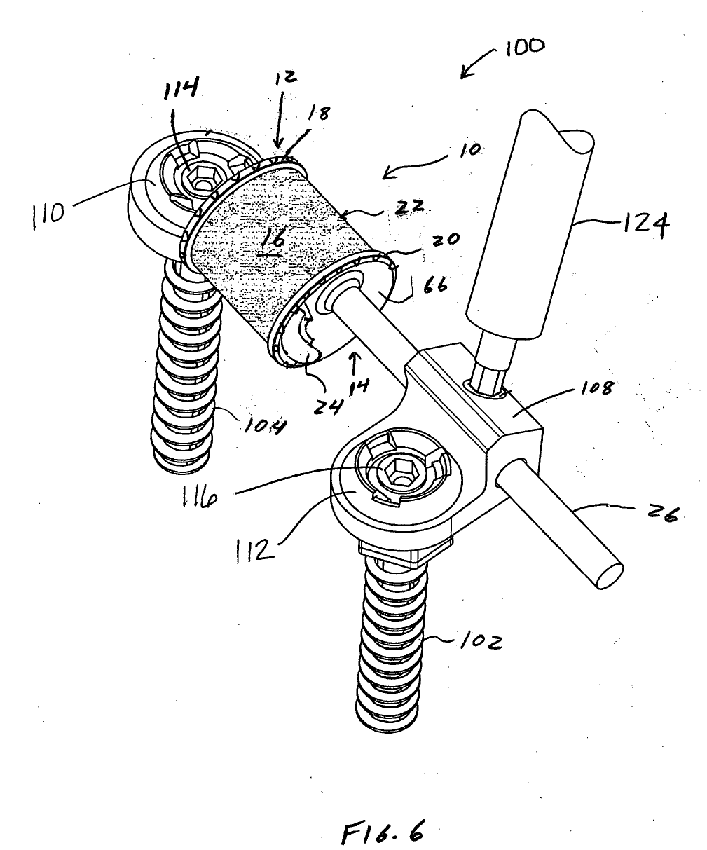

Turning to FIGS. 5A-5C, an exemplary sheath assembly 22 according to the present disclosure is depicted. Sheath assembly 22 includes sheath member 16, first connector ring 18 and second connector ring 20. Sheath member 16 is substantially cylindrical in geometry and defines opposed, substantially circular edges. Sheath member 16 is fabricated from a material that accommodates relative movement of end caps 12, 14, while exhibiting substantially inert behavior relative to surrounding anatomical structures. The sheath member 16 is generally fabricated from expanded polytetrafluoroethylene, ultra-high molecular weight polyethylene, a copolymer of polycarbonate and a urethane, or a blend of a polycarbonate and a urethane. Additional disclosure with respect to exemplary sheath member design, function and operation for use according to the present disclosure is provided in a co-pending U.S. patent application entitled “Surgical Implant Devices and Systems Including a Sheath Member” (Ser. No. 11/027,073; filed Dec. 31, 2004), the contents of which are hereby incorporated by reference.

As most clearly depicted in FIG. 5B, the opposed edges of sheath member 16 are received within the internal cavities 46 defined by connector rings 18, 20 and, once positioned therein, connector rings 18, 20 are crimped, compressed or swaged so as to secure sheath member 16 relative to connector rings 18, 20, respectively. According to exemplary embodiments of the present disclosure, deflected edges 52 of the respective connector rings 18, 20 assist in guiding sheath member 16 into the associated internal cavity and retaining the sheath member edges therein pending the crimping, compression or swaging operation. As noted above, alternative and/or additional steps may be taken to temporarily retain the positioning of sheath member 16 relative to connector rings 18, 20, e.g., an adhesive may be added to the edges of sheath member 16 and/or internal cavities 46 of connector rings 18, 20.

Of note, notches 44 advantageously facilitate the fabrication of sheath assembly 22 according to the present disclosure. More particularly, aperture regions 48 facilitate the compression of connector rings 18, 20 into secure engagement with sheath member 16. Indeed, aperture regions 48 permit the outer walls 42 of the respective connector rings 18, 20 to assume a reduced diameter as the connector rings are crimped, compressed or swaged with respect to sheath member 16, thereby avoiding any distortional effect (e.g., a “bottle cap effect” that may result in discontinuous crimping/swaging of the sheath member within the connector ring). The aperture regions 48 also provide regions that may be occupied by the sheath member 16 when the connector rings 18, 20 are crimped, compressed or swaged relative thereto, and further provide an ability to visually confirm that the sheath member 16 is properly positioned within connector ring 18, 20. “Visual confirmation” may also be achieved through appropriate sensor equipment, e.g., in an automated fashion, as will be readily apparent to persons skilled in manufacturing techniques. A small portion of the sheath member 16 may protrude through one or more aperture regions 48, depending on the compression force applied and/or the positioning of the sheath member within internal cavity 46.

Once sheath assembly 22 is formed, the sheath member 16 and connector rings 16, 18 may be advantageously handled as a unit, thereby enhancing inventory operations and further assembly steps. Thus, returning to FIGS. 1A, 1B and 2B, sheath assembly 22 may be mounted with respect to other components or subassemblies of a spinal stabilization device. In exemplary embodiments of the present disclosure, the sheath assembly 22 is positioned over or around spring members 24, 34, with connector rings 18, 20 in a substantially concentric orientation with respect to flange portions 60, 62 of end caps 12, 14, respectively. As shown in FIG. 2B, flange portions 60, 62 may be provided with annular channels 64, 66, respectively. In assembling sheath assembly 22 to the subassembly shown in FIGS. 2A and 2B, the connector rings 18, 20 are generally concentrically positioned with respect to the flange portions 60, 62 (and preferably the annular channels 64, 66 thereof), and the connector rings 16, 18 are further crimped, compressed or swaged with respect thereto. Further deformation of connector rings 16, 18 is accommodated by radially-spaced notches 44, and the sheath assembly 22 is thereby fixedly mounted with respect to the underlying subassembly. According to exemplary embodiments of the present disclosure, sheath assembly 22 is mounted with respect to the underlying subassembly, e.g., the flange portions 60, 62 of end caps 12, 14, such that there are substantially no gaps and substantially no rotation therebetween. Alignment operations of sheath member 16 relative to end caps 18, 20, and secure mounting therebetween, are substantially benefited by the pre-fabrication of sheath assembly 22, as described herein.

With reference to FIG. 6, an exemplary system 100 including the disclosed spinal stabilization device 10 (which, in turn, includes sheath assembly 22) and a pair of pedicle screws 102, 104 is schematically depicted. Cooperation between the spinal stabilization device 10 and the pedicle screws 102, 104 is facilitated by ball/spherical elements 110, 112, and set screws 114, 116. In the exemplary system of FIG. 6, the ball/spherical element 110 cooperates with the head of pedicle screw 104 such that a global/dynamic joint is formed therebetween. The set screw 114 is inserted into the head of the pedicle screw 106, thereby securing the head of the pedicle screw 102 within the ball/spherical element 110.

Turning to the opposite end of exemplary system 100, attachment member 108 is configured to receive ball/spherical element 112. The ball/spherical element 112 receives the head of pedicle screw 102, such that a global/dynamic joint is formed therebetween. Set screw 116 is inserted into the head of the pedicle screw 102 , thereby securing the head of pedicle screw 102 within the ball/spherical element 112. Rod 26 is configured to be inserted into the attachment member 108 which may include, for example, a transverse aperture to accommodate rod 26, and a set screw is generally used to secure the rod at a desired position, e.g., using driver 124.

Exemplary system 100 advantageously provides dynamic stabilization in clinical applications based on the dynamic properties of spinal stabilization device 10. Moreover, sheath member 16 provides a protective casing to the spring elements positioned therewithin, while simultaneously accommodating relative movement between end caps 14, 16. The design and operation of spring connectors 18, 20 facilitate efficient and reliable assembly, while also ensuring security of sheath member 16 relative to the underlying structures during in situ applications.

Although the present disclosure has been disclosed with reference to exemplary embodiments and implementations thereof, those skilled in the art will appreciate that the present disclosure is susceptible to various modifications, refinements and/or implementations without departing from the spirit or scope of the present invention. In fact, it is contemplated the disclosed sheath assembly may be employed in a variety of environments and clinical settings without departing from the spirit or scope of the present invention. Accordingly, while exemplary embodiments of the present disclosure have been shown and described, it will be understood that there is no intent to limit the invention by such disclosure, but rather, the present invention is intended to cover and encompass all modifications and alternate constructions falling within the spirit and scope hereof.

1. A sheath assembly comprising:
a. a sheath member;
b. at least one connector ring secured with respect to the sheath member, the at least one connector ring defining a circumference and including a plurality of radially-spaced notches positioned around the circumference.

2. A sheath assembly according to claim 1, wherein the sheath member is substantially cylindrical.

3. A sheath assembly according to claim 1, wherein the sheath member is fabricated from a material selected from the group consisting of expanded polytetrafluoroethylene, ultra-high molecular weight polyethylene, a copolymer of polycarbonate and a urethane, and a blend of a polycarbonate and a urethane.

4. A sheath assembly according to claim 1, wherein the at least one connector ring defines a substantially V-shaped cross-section before being secured to the sheath member.

5. A sheath assembly according to claim 1, wherein each of the plurality of radially-spaced notches includes an aperture region.

6. A sheath assembly according to claim 5, wherein each of the plurality of radially-spaced notches further includes a notch finger.

7. A sheath assembly according to claim 1, wherein the at least one connecting ring includes an inner face, an apex region and an outer wall, and wherein the plurality of radially-spaced notches are formed at least in part in the apex region.

8. A sheath assembly according to claim 7, wherein the inner face defines a deflected edge.

9. A sheath assembly according to claim 8, wherein the deflected edge is substantially parallel to the outer wall.

10. A sheath assembly according to claim 1, wherein the at least one connector ring includes a pair of connector rings, and wherein a first connector ring is secured to a first end of the sheath member and a second connector ring is secured to a second end of the sheath member.

11. A sheath assembly according to claim 1, wherein the at least one connector ring defines an interior cavity and a first end of the sheath member extends into the internal cavity.

12. A sheath assembly according to claim 1, wherein the at least one connector ring is secured with respect to the sheath member by crimping, compression or swaging.

13. A sheath assembly comprising:
a. a sheath member;
b. at least one connector ring secured with respect to the sheath member, the at least one connector ring including an inner cylindrical face, a substantially circular apex region and an angularly oriented outer wall,
wherein the at least one connector ring defines a substantially V-shaped cross-section that is configured and dimensioned to receive an end of the sheath member; and
wherein the inner cylindrical face is adapted to shield the end of the of the sheath member from one or more elements positioned therewithin.

14. A sheath assembly according to claim 13, wherein said at least one connector ring further comprises a plurality of radially-spaced notches.

15. A spinal stabilization device, comprising:
a. first and second end caps in spaced relation;
b. at least one spring member positioned between the first and second end caps; and
c. a sheath assembly mounted with respect to the first and second end caps and around the at least one spring member, the sheath assembly including a sheath member and at least one connector ring secured with respect to the sheath member, the at least one connector ring defining a circumference and including a plurality of radially-spaced notches positioned around the circumference.

16. A spinal stabilization system 15, wherein the sheath member is fabricated from a material selected from the group consisting of expanded polytetrafluoroethylene, ultra-high molecular weight polyethylene, a copolymer of polycarbonate and a urethane, and a blend of a polycarbonate and a urethane.

17. A spinal stabilization system according to claim 15, wherein the at least one connector ring defines a substantially V-shaped cross-section before being secured to the sheath member.

18. A spinal stabilization system according to claim 15, wherein each of the plurality of radially-spaced notches includes an aperture region.

19. A spinal stabilization system according to claim 18, wherein each of the plurality of radially-spaced notches further includes a notch finger.

20. A spinal stabilization system according to claim 15, wherein the at least one connecting ring includes an inner face, an apex region and an outer wall, and wherein the plurality of radially-spaced notches are formed in the outer wall.

21. A spinal stabilization system according to claim 20, wherein the inner face defines a deflected edge.

22. A spinal stabilization system according to claim 21, wherein the deflected edge is substantially parallel to the outer wall.

23. A spinal stabilization system according to claim 15, wherein the at least one connector ring includes a pair of connector rings, and wherein a first connector ring is secured to a first end of the sheath member and a second connector ring is secured to a second end of the sheath member.

24. A spinal stabilization system according to claim 15, wherein the at least one connector ring defines an interior cavity and a first end of the sheath member extends into the internal cavity.

25. A spinal stabilization system according to claim 15, wherein the at least one connector ring is secured with respect to the sheath member by crimping, compression or swaging.

26. A spinal stabilization system according to claim 15, wherein the sheath assembly is secured with respect to the first and second end caps by crimping, compression or swaging.

27. A method for assembling a sheath assembly, comprising:
a. providing a sheath member;
b. providing a first connector ring that includes an inner face, an apex region, an outer wall and a plurality of radially-spaced notches formed at least in part in the apex region, wherein the first connector ring defines an internal cavity;
c. positioning an end of the sheath member within the internal cavity; and
d. securing the sheath member with respect to the first connector ring by crimping, compression or swaging.

28. A method according to claim 27, further comprising:
a. providing a second connector ring that includes an inner face, an apex region, an outer wall and a plurality of radially-spaced notches formed at least in part in the apex region, wherein the second connector ring defines a second internal cavity;
b. positioning an opposite end of the sheath member within the second internal cavity; and
c. securing the sheath member with respect to the second connector ring by crimping, compressing or swaging.

29. A method according to claim 28, further comprising securing the sheath member to first and second end caps of a spinal stabilization device.

30. A method according to claim 29, wherein the sheath member is secured with respect to the spinal stabilization device by crimping, compressing or swaging of the first connector ring with respect to the first end cap, and crimping, compressing or swaging of the second connector ring with respect to the second end cap.
